(19) United States
(12) Patent Application Publication
(10) Pub. No.: US 2026/0256508 A1
(43) Pub. Date: Sep. 3, 2026

(54) FLUIDLY CONNECTED PLASMA CATHETER SYSTEM AND METHODS THEREOF

(71) Applicant: ASAHI INTECC CO., LTD., Seto-shi (JP)

(72) Inventors: Wayne OGATA, San Ramon, CA (US); Takeshi MOGI, Tustin, CA (US)

(73) Assignee: ASAHI INTECC CO., LTD., Seto-shi (JP)

(21) Appl. No.: 19/654,825

(22) Filed: Apr. 22, 2026

Related U.S. Application Data
(63) Continuation of application No. PCT/JP2024/035610, filed on Oct. 4, 2024.
(60) Provisional application No. 63/545,511, filed on Oct. 24, 2023.

(51) Int. Cl. A61B 18/04; A61B 18/00

(52) U.S. Cl. CPC A61B 18/04; A61B 2018/00077; A61B 2018/00083; A61B 2018/00178; A61B 2018/00184

(57) ABSTRACT

A plasma catheter system includes a power connection system that detachably couples an energy source to a connection hub, a first longitudinal member having an inner lumen and a distal electrode, and a second longitudinal member having a tip electrode. The second longitudinal member inserts through the inner lumen and is freely movable within it, forming a bipolar arrangement between the two electrodes. A conductive fluid in the connection hub electrically couples the second longitudinal member to the energy source without restricting relative movement.

### CROSS REFERENCE TO RELATED APPLICATIONS

This application is a Bypass Continuation of PCT/JP2024/035610 filed on October 4, 2024, which claims priority to U.S. provisional patent application 63/545,511 filed October 24, 2023. The contents of these applications are incorporated herein by reference in their entirety.

### TECHNICAL FIELD

This technology relates to fluidly connected plasma catheter systems and methods thereof.

### Background Art

FIG. 1 is a diagram of an example of a prior art catheter system.

### Summary

The present fluidly connected plasma catheter system includes: a power connection system configured to detachably couple an energy source to a connection hub; a first longitudinal member coupled to the connection hub and configured to move along a peripheral vessel of a patient, wherein the first longitudinal member extends along a first length between a first proximal end and a first distal end and wherein the first longitudinal member includes: an inner lumen extending along the first length of the first longitudinal member; and a distal electrode located on an outer surface of the first longitudinal member proximate to the first distal end and electrically coupled to the energy source by the detachable coupling of the power connection system to the connection hub; and a second longitudinal member configured to insert into the peripheral vessel through the lumen of the first longitudinal member, the second longitudinal member extending along a second length between a second proximal end and a second distal end comprising a tip electrode, wherein the second longitudinal member is freely movable within the lumen of the first longitudinal member and creates a bipolar arrangement between the distal electrode and the tip electrode to deliver energy while remaining electrically coupled to the energy source by a conductive fluid in a fluid connection system in the connection hub.

### BRIEF DESCRIPTION OF DRAWINGS

FIG. 1 is a diagram of an example of a prior art catheter system;

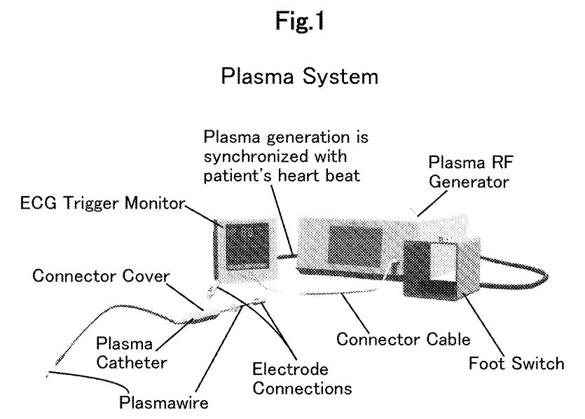

FIG. 2 is a diagram of an example of a fluidly connected plasma catheter system;

FIG. 3 is a perspective view of a connection hub to a catheter in the fluidly connected plasma catheter system;

FIG. 4 are cross-sectional views of an example of the connection hub and of the catheter with the plasma wire coupled by a conductive fluid; and

FIG. 5 is a cross-sectional view of an example of the fluid connection system in a connection hub of the fluidly connected plasma catheter system.

### DESCRIPTION OF EMBODIMENTS

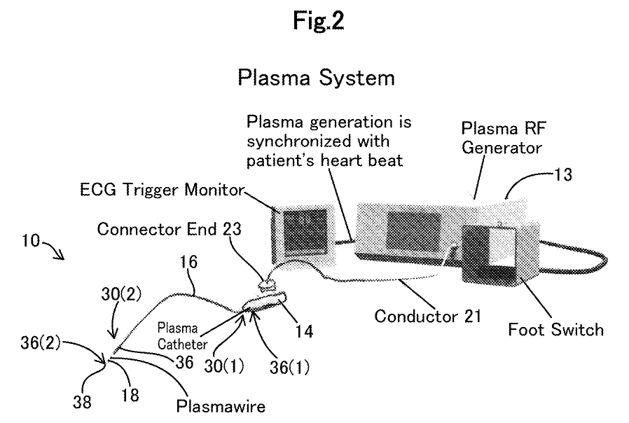

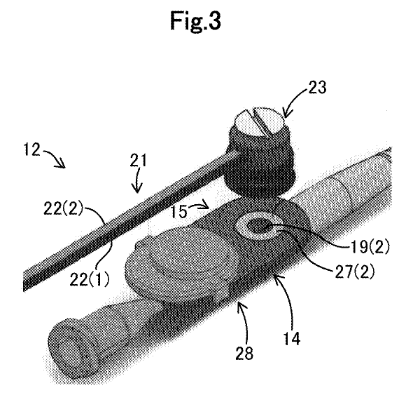

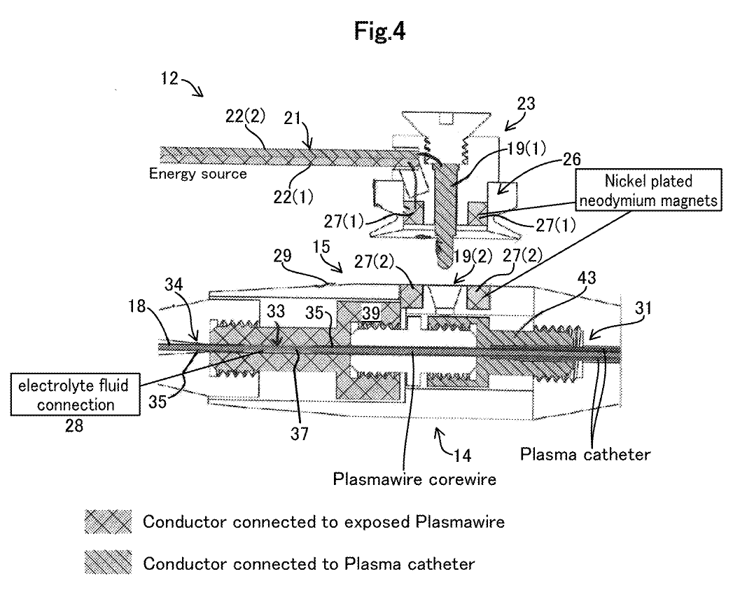

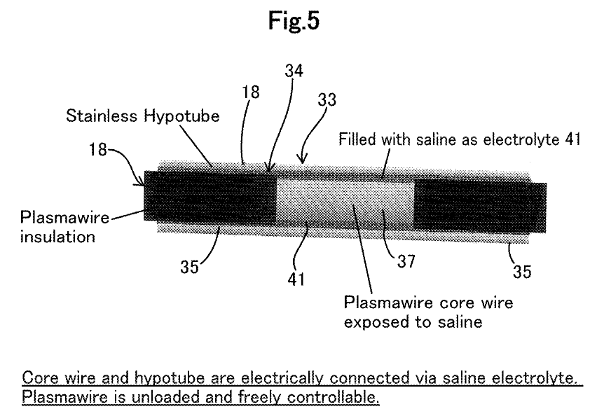

An example of a fluidly connected plasma catheter system 10 is illustrated in FIGS. 2-5. In this example, the fluidly connected plasma catheter system 10 includes a power connection system 12, a connection hub 14, a first longitudinal member 16 and a second longitudinal member 18, although the system may have other types and/or numbers of other systems, devices, components, or other elements which may be in other combinations. Examples of this technology provide a number of advantages including providing a fluidly connected plasma catheter system that facilitates an easier connection and ease of use by an operator.

The power connection system 12 is configured to detachably couple an energy source 13 to the connection hub 14. In this example, the power connection system 12 includes a conductor 21 comprising conductive leads 22(1) and 22(2) detachably coupled at one end to a connector end 23 and at another end to an energy source 13, although the power connection system 12 may comprise other types and/or numbers of other components or other elements which may be in other configurations. The connector end 23 is configured to detachably couple the conductive leads 22(1) and 22(2) to first magnetic conductive element 27(1) and male conductive connector 19(1), respectively with a magnetic connection system 26, although other types of detachable connection systems can be used. The male conductive connector 19(1) in the connector end has a shape configured to detachably mate and couple to a female conductive connector 19(2) in the connection hub 14, although other types of coupling mechanisms which may be in other configurations may be used.

The magnetic connection system 26 comprises the first magnetic conductive element 27(1) in the connector end 23 with a polarity configured to detachably couple with a second magnetic conductive element 27(2) in the connection hub 14, although the magnetic connection system can comprise other numbers of magnetic conductive elements in other configurations. In this example, the first and second magnetic conductive elements 27(1) and 27(2) of the magnetic connection system 26 each comprise a ring-shaped conductive magnetic terminal, although other types and/or numbers of conductive terminals which may be in other configurations could be used.

The connection hub 14 includes a hub housing 15 which houses a portion of the first longitudinal member 16 and the second longitudinal member 18, the female connector 19(2), the second magnetic conductive element 27(2) of the magnetic connection system 26, and a fluid connection system 28, although the connection hub can comprise other types and/or numbers of other components or other elements which may be in other configurations. The hub housing 15 secures the female connector 19(2) adjacent an outer surface 29 in a position to detachably couple and mate with the male connector 19(1) of the connector end 23. The female connector 19(2) in the connection hub 14 is coupled by a conductive connection element 43 in the hub housing 15 to the first longitudinal member 16 which is made of a conductive material and is coupled to a distal electrode 36 located proximate to a first distal end 30(2) of the first longitudinal member 16, although other types and configurations of connections may be used. The hub housing 15 also secures the second magnetic conductive element 27(2) adjacent the outer surface 29 in a position to detachably connect and couple with the first magnetic conductive element 27(1) of the connector end 23.

The hub housing 15 also defines a passage 31 which extends longitudinally through the hub housing 15 and is configured to receive and secure a portion of the first longitudinal member 16. Further, the hub housing 15 defines a chamber 33 for the fluid connection system 28 in this passage 31 which is configured to receive the second longitudinal member 18 comprising a plasmawire where at least a portion 37 of the surrounding insulation 35 has been removed. The chamber 33 is shaped to hold a conductive fluid 41, such as a saline electrolyte or blood by way of example only. When the conductive fluid 41 is introduced it is used to couple the portion 37 to a conductive connection element 39 that forms part of the hub housing 15 (and also defines the chamber 33) which is coupled to the magnetic conductive element 27(2) and then is detachably coupled to the magnetic conductive element 27(1) that is coupled to the conductive lead 22(1).

The first longitudinal member 16 is coupled to the connection hub 14 and is configured to have a size and dimensions to move along a peripheral vessel of a patient. The first longitudinal member 16 extends along a first length between a first proximal end 30(1) and a first distal end 30(2). In this example, the first longitudinal member 16 comprises a hypotube 32 which defines a lumen 34 that extends along the first length of the first longitudinal member 16. A distal electrode 36 is located on an outer surface of the first longitudinal member 16 proximate to the first distal end 30(2) and is electrically coupled to the conductive connection element 43 which is coupled to the female connector 19(2) that is detachably coupled to the male connector 19(1) which is coupled to the conductive lead 22(2).

The second longitudinal member 18 is configured to have a size and dimensions to insert into and through the lumen 34 of the first longitudinal member 16. The second longitudinal member 18 extends along a second length between a second proximal end 36(1) and a second distal end 36(2) with a tip electrode 38. With the fluid connection system 28, the second longitudinal member 18 is more freely movable within the lumen 34 of the first longitudinal member 16 while still remaining coupled to create a bipolar arrangement between the distal electrode 36 and the tip electrode 38 when the energy source 13 is engaged by a switch or other mechanism to couple power by the detachable coupling of the connector end 23 and the connection hub 14.

Having thus described the basic concept of the disclosure, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the disclosure. Accordingly, the disclosure is limited only by the following claims and equivalents thereto.

1. A fluidly connected plasma catheter system comprising:
a power connection system configured to detachably couple an energy source to a connection hub;
a first longitudinal member coupled to the connection hub and configured to move along a peripheral vessel of a patient, wherein the first longitudinal member extends along a first length between a first proximal end and a first distal end and wherein the first longitudinal member comprises:
an inner lumen extending along the first length of the first longitudinal member; and
a distal electrode located on an outer surface of the first longitudinal member proximate to the first distal end and electrically coupled to the energy source by the detachable coupling of the power connection system to the connection hub; and
a second longitudinal member configured to insert into the peripheral vessel through the inner lumen of the first longitudinal member, the second longitudinal member extending along a second length between a second proximal end and a second distal end comprising a tip electrode, wherein the second longitudinal member is freely movable within the inner lumen of the first longitudinal member and creates a bipolar arrangement between the distal electrode and the tip electrode to deliver energy while remaining electrically coupled to the energy source by a conductive fluid in a fluid connection system in the connection hub.

2. The system as set forth in claim 1 wherein
the power connection system comprises:
a connector end that detachably couples to the connection hub and
conductive leads that detachably couple the connector end to the energy source.

3. The system as set forth in claim 2 wherein
the connector end further comprises a magnetic connection system that detachably couples the connector end to the connection hub.

4. The system as set forth in claim 1 wherein
the inner lumen in the first longitudinal member is in fluid communication with the fluid connection system in the connection hub.

5. The system as set forth in claim 1 wherein
the second longitudinal member further comprises at least one outer insulation layer about an outer surface of an inner conductive element,
wherein at least a portion of the conductive element is exposed in the fluid connection system in the connection hub.

6. The system as set forth in claim 1 wherein
the conductive fluid further comprises an electrolyte fluid.

7. A method for making a fluidly connected plasma catheter system comprising:
providing a power connection system configured to detachably couple an energy source to a connection hub;
coupling a first longitudinal member to the connection hub, wherein the first longitudinal member is configured to move along a peripheral vessel of a patient, wherein the first longitudinal member extends along a first length between a first proximal end and a first distal end and wherein the first longitudinal member comprises:
an inner lumen extending along the first length of the first longitudinal member; and
a distal electrode located on an outer surface of the first longitudinal member proximate to the first distal end and electrically coupled to the energy source by the detachable coupling of the power connection system to the connection hub; and
providing a second longitudinal member configured to insert into the peripheral vessel through the inner lumen of the first longitudinal member, the second longitudinal member extending along a second length between a second proximal end and a second distal end comprising a tip electrode, wherein the second longitudinal member is freely movable within the inner lumen of the first longitudinal member and creates a bipolar arrangement between the distal electrode and the tip electrode to deliver energy while remaining electrically coupled to the energy source by a conductive fluid in a fluid connection system in the connection hub.

8. The method as set forth in claim 7 wherein
the providing the power connection system further comprises:
providing a connector end that detachably couples to the connection hub and conductive leads that detachably couple the connector end to the energy source.

9. The method as set forth in claim 8 wherein
the connector end further comprises a magnetic connection system that detachably couples the connector end to the connection hub.

10. The method as set forth in claim 7 wherein
the inner lumen in the first longitudinal member is in fluid communication with the fluid connection system in the connection hub.

11. The method as set forth in claim 7 wherein
the second longitudinal member further comprises at least one outer insulation layer about an outer surface of an inner conductive element,
wherein at least a portion of the conductive element is exposed in the fluid connection system in the connection hub.

12. The method as set forth in claim 7 wherein
the conductive fluid further comprises an electrolyte fluid.
